(19) United States
(12) Patent Application Publication
(10) Pub. No.: US 2005/0107610 A1
(43) Pub. Date: May 19, 2005

(54) Synthesis of 2-chloro-3,6-dialkyl pyrazines

(75) Inventor: David Warner Rappath, Kalamazoo, MI (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/990,113

(22) Filed: Nov. 15, 2004

Related U.S. Application Data
(60) Provisional application No. 60/523,499, filed on Nov. 19, 2003.

(51) Int. Cl. C07D 241/04

(52) U.S. Cl. 544/336; 544/385

(57) ABSTRACT

A process for the production of 3,6-dialkyl-2,5-piperazinediones is disclosed.

This application claims priority under 35 U.S.C 119 of U.S. Provisional 60/523,499 filed Nov. 19, 2003. The entire contents of the prior application are incorporated herein by reference.

### FIELD OF INVENTION

This invention relates to method for synthesizing 2-chloro-3,6-dialkyl pyrazines. 2-Chloro-3,6-dialkyl pyrazines are intermediates in the synthesis of substituted aryl pyrazines, which are useful as CRF receptor modulators capable of treating a wide variety of neurological disorders.

### BACKGROUND OF THE INVENTION

WO 01/60806 discloses a synthesis of 2-chloro-3,6-dialkyl pyrazines according to a know literature procedure starting with 2-alkyl glycine (Chemical and Pharmaceutical Bulletin of Japan 1979, 27,2027). The method uses a mixture of POCl₃ and PCl₅ and is heated in a sealed tube at 140° C. to gives a 38% yield. Accordingly, there is a need for a synthesis of 2-halo-3,6-dialkyl pyrazines in high yield.

### SUMMARY OF THE INVENTION

### DETAILED DESCRIPTION OF THE INVENTION

We have found that 2-chloro-3,6-dialkyl pyrazines may be synthesized is a high yield in a process according to Scheme I.

In the first step an amino acid or amino acid ester is condensed to form a 3,6-dialkyl-2,5-piperazinedione of Formula I.

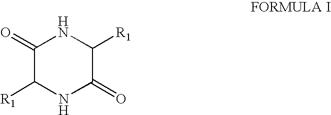

The 3,6-dialkyl-2,5-piperazinedione is subsequently reacted with phosphorous oxychloride to form a 2-chloro-3,6-dialkyl pyrazine Formula II.

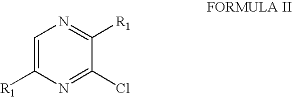

Surprisingly, it has been found that after the first step, the addition of 3 to 8 percent water by volume to the original amount of ethylene glycol improves the crystallization of the 3,6-dialkyl-2,5-piperazinedione, thereby enhancing the recovery of the product.

It was also discovered that in the chlorination step the addition of 1 to 2 moles of Me₄NCl per mole of 3,6-dialkyl-2,5-piperazinedione lowers the amount of 2,5-dichloro-3,6-dialkyl pyrazine produced as an undesirable impurity. In addition, it was discovered that lowering the temperature of chlorination step lowers the level of solvent chlorination products. The process is illustrated in Scheme I:

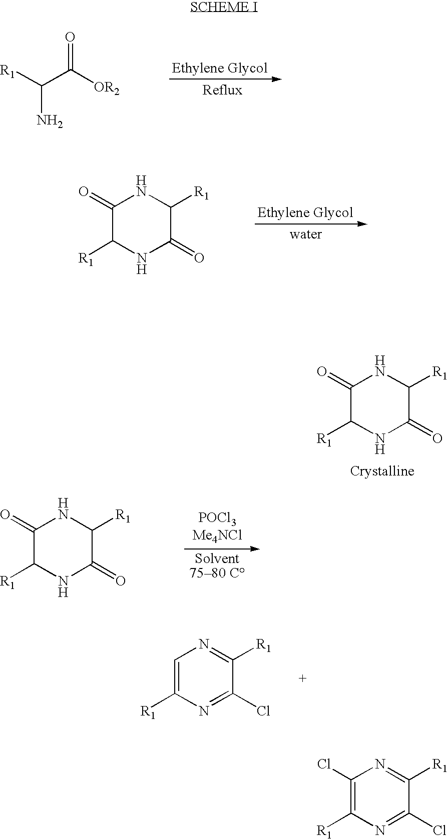

In Scheme I R₁ is a C₁-C₆ alkyl group, R₂ is H, or a C₁-C₆ alkyl group, and Me is a methyl group.

The first step of the reaction, that is the condensation of the amino acid or amino acid ester to form a 3,6-dialkyl-2,5-piperazinedione, is conducted at atmospheric pressure at the reflux temperature of ethylene glycol (about 198° C.). During the first step of the reaction water or alcohol is formed depending upon whether an amino acid or amino acid ester is being condensed. The water or alcohol produced is retained in the reaction mixture. Although the reaction proceeds well under these conditions, there is a tendency for the 3,6-dialkyl-2,5-piperazinedione form a slurry that is difficult to filter. Accordingly, after the reaction is complete, it is preferred to allow the reaction mixture to cool to a temperature between 120° C. and 140° C., at which point between 3 and 8 volume percent water based upon the amount of ethylene glycol, is added. Having the reaction mixture at a temperature of 130° C. and 135° C. provides good results. Adding water at a level of 5 volume percent water based upon the amount of ethylene glycol also provides good results. The reaction mixture is then allowed to cool to room temperature (18-25° C.). The addition of water results in the formation of a slurry that may be more readily filtered.

The 3,6-dialkyl-2,5-piperazinedione may be further reacted with POCl₃ in a suitable solvent in the presence tetramethyl ammonium chloride (Me₄NCl). One to two moles of tetramethyl ammonium chloride are used for every mole of 3,6-dialkyl-2,5-piperazinedione. The Me₄NCl has an important role in the reaction in that it greatly reduces the formation of the dichloro-pyrazine impurity. The presence of tetramethyl ammonium chloride reduces the amount of 2,5 dichloropyrazine produced to less than 5% of the final product.

The solvent for the second step may be selected from the group consisting of inert inorganic solvents boiling in the range from approximately 75° C. to 115° C. These inert solvents include hydrocarbons, chlorinated hydrocarbons, and ethers. Examples of such solvents include heptane, cyclohexane, benzene, toluene, carbon tetrachloride, dichlopropane isomers, propyl ether, and dioxane.

Even somewhat inert solvent such as the dioxane undergo some chlorination. When dioxane is selected as the solvent this side reaction tends to produce dichloroethyl ether (DCEE). The side reaction is less likely when hydrocarbon or chlorinated hydrocarbon solvents are selected.

After the conversion of the 3,6-dialkyl-2,5-piperazinedione to 2-chloro-3,6-dialkyl pyrazine is complete, it is important that the reaction mixture be quenched properly in order to assure a high yield of 2-chloro-3,6-dialkyl pyrazine. The product, the 2-chloro-3,6-dialkyl pyrazine is somewhat unstable under the acid conditions.

If the reaction mixture is quenched in water, the hydrolysis of excess POCl₃ generates heat, HCl and H₃PO₄ both of which are strong acids. This hot acid solution can cause the hydrolysis of the 2-chloro-3,6-dialkyl pyrazine and thereby result in a loss of yield. Accordingly, it is preferred to quench the reaction mixture in an aqueous basic solution. Suitable bases for the aqueous basic solution for quenching include LiOH, LiHCO₃, Li₂CO₃, Na₃PO₄, NaOH, NaHCO₃, Na₂CO₃, Na₃PO₄, KOH, KHCO₃, K₂CO₃, K₃PO₄, Na⁺, K⁺, Mg(OH)₂, Mg(HCO₃)₂, MgCO₃, Mg₃(PO₄)₂, Ca(OH)₂, Ca(HCO₃)₂, CaCO₃, and Ca₃(PO₄)₂. The preferred aqueous basic solution quenching is 10-15% NaOH.

### EXAMPLE 1

### 3,6-diethyl-2,5-piperazinedione

1.5 Kg of 2-aminobutyric acid followed by 8 L of ethylene glycol was charged to a 22 L 5 neck round bottom flask equipped with: an electric heating mantle, over-head stirrer, two friedrichs style reflux condensers, under nitrogen. The mixture was agitated by hand until it was sufficiently suspended to use the over-head stirrer. 1.0 kg of 2-aminobutyric acid was added to the suspension followed by 2 L of ethylene glycol. (A single addition is possible with a more powerful stirrer.) The mixture was heated to 170 to 180° C. and stirred for 6 hours. The reaction mixture was cooled to 130 to 135° C., 500 ml of water was added over 15 minutes, and the resulting mixture was cooled to 20 to 30° C. over 12 hours. The 3,6-diethyl-2,5-piperazinedione slurry was filtered, and the reaction vessel was rinsed with 500 ml of cold ethanol. The product cake was rinsed 1×2 L, 1×3 L of cold water, and 1×3 L of cold ethanol. The material was dried to constant weight, c.a. 4.5 hours, in a 130° C. vacuum oven.

The yield was 2.1 kg of 3,6-diethyl-2,5-piperazinedione as a white crystalline solid. This is 104% of the theoretical yield and indicates that the material contained about 4% impurities. ¹H NMR (400 MHz, DMSO-d₆) δ 0.8329 (q, J=7 Hz, 6H), 1.6941 (h, J=7 Hz, 4H), 3.7866 (d, J=6 Hz, 2H), 8.0687 (br s, 2H);

### EXAMPLE 2

### 2-chloro-3,6-diethyl Pyrazine

900.00 G of 3,6-diethyl-2,5-piperazinedione followed by 753.42 g of Me₄NCl, 4.5 l of dioxane, and 2.4 l. of POCl₃ was charged to a 22 L 5 neck round bottom flask equipped with: and electric heating mantle, over-head stirring, and two laboratory gas scrubbers. The reaction mixture was covered under nitrogen. The reaction mixture was warmed to 65° C. Over the course of an hour, the reaction was heated to 75° C. in 5° C. increments. The reaction was stirred for 4 hours and monitored by gas liquid chromatography (GLC) in order to determine the degree of completion. After 4 hours, less than 5% intermediate remained at which point the reaction was deemed to be complete.

The reaction mixture was allowed to cool overnight to 20° C. to 25° C. The reaction was quenched in two aliquots of approximately 4.3 liters each. Each aliquot was quenched over 2 hours in 7.3 L of 12% NaOH that had been cooled to 0 to 5° C. The temperature was maintained at less than 60° C. After the addition of the reaction mixture to the 12% aqueous NaOH was complete, the solution was stirred for 1 hour. The resulting slurry was filtered through a pad of celite, rinsing with 4.5 L of 2:1 EtOAc:Hex (Ethyl Acetate:Hexane). The filtrate had an organic phase, and an aqueous phase. The phases were separated. The organic phase was washed three times with 2.5 L of 1N HCl. Following the acid wash, the organic phase was washed twice with 2.5 L of 1N NaOH.

The organic phase for each aliquot was evaporated under reduced pressure, using a rotary evaporator, to produce an oil. The oil from the two aliquots were combined and poured directly onto a 2 kg pad of silica gel. The pad was rinsed with 4.4 L of 10:90 EtOAc(Ethyl Acetate): mixed octanes, that is, a mixture of octane isomers and the organic solution was evaporated under reduced pressure, using a rotary evaporator, to produce an oil. This yield was 808 g of yellow oil, yield 88%.

¹H NMR (400 MHz, CDCl₃) δ 1.300 (t, J=6 Hz, 6H), 2.791 (q, J=8 Hz, 2H), 2.926 (q, J=7 Hz, 2H), 8.297 (s, 1H).

1. A process for purifying a reaction mixture containing a 3,6-dialkyl-2,5-piperazinedione formula I in ethylene glycol comprising the steps of:
bringing the reaction mixture to a temperature of from 120° C. to 140° C.,
adding to the reaction mixture 3 to 8 volume percent water based upon the amount of ethylene glycol to form a reaction mixture containing added water,
cooling the reaction mixture containing added water to a temperature of 18° C. to
25° C., filtering the reaction mixture containing added water to collect the precipitate of 3,6-dialkyl-2,5-piperazinedione.

2. A process according to claim 1 in which the reaction mixture is brought to a temperature of 130° C. to 135° C.

3. A process according to claim 1 in which water is added to the reaction mixture at a level of 5 volume percent water based upon the amount of ethylene glycol.

4. A process according to claim 1 comprising the additional step of condensing an amino acid of the following formula: 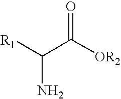
wherein R₁ is a C₁-C₆ alkyl group, R₂ is H, or a C₁-C₆ alkyl group, by placing the amino acid in ethylene glycol and allowing the ethylene glycol to reflux until the reaction is complete, thereby forming a reaction mixture containing 3,6-dialkyl-2,5-piperazinedione in ethylene glycol.

5. A process for the preparation of 2-chloro-3,6-dialkyl pyrazines comprising the steps of:
reacting a 3,6-dialkyl-2,5-piperazinedione of Formula II with POCl₃ in the presence of 1 to 2 moles of Me₄NCl per mole of 3,6-dialkyl-2,5-piperazinedione in a suitable solvent;
quenching the reaction mixture in an aqueous basic solution.

6. A process according to claim 5 in which the aqueous basic solution comprises a solution of 10-15% NaOH in water.

7. A process according to claim 4 comprising the additional steps of reacting a 3,6-dialkyl-2,5-piperazinedione of Formula II with POCl₃ in the presence of 1 to 2 moles of Me₄NCl per mole of 3,6-dialkyl-2,5-piperazinedione in a suitable solvent; and quenching the reaction mixture in an aqueous basic solution to produce a slurry.

8. A process according to claim 7 further comprising the steps of:
filtering the slurry of claim 7 through a pad of celite;
rinsing the material retained on the celite pad with 4.5 L of 2:1 Ethyl Acetate:Hexane;
washing the organic phase of the filtrate, three times, with 2.5 L of 1N HCl;
washing, the organic phase, twice, with 2.5 L of 1N NaOH;
evaporated the organic phase for each aliquot under reduced pressure, to produce an oil;
pouring the oil from the two aliquots onto a 2 kg pad of silica gel;
rinsing the pad with 4.4 L of 10:90 Ethyl Acetate: mixed octanes;
and evaporating the organic solution under reduced pressure to produce an oil comprising a 3,6-dialkyl-2,5-piperazinedione of Formula I.
